(19) United States
(12) Patent Application Publication
(10) Pub. No.: US 2022/0160625 A1
(43) Pub. Date: May 26, 2022

(54) METHODS AND DEVICES FOR REDUCING INTRAOCULAR OXIDATIVE DAMAGE

(71) Applicant: THE REGENTS OF THE UNIVERSITY OF COLORADO, A BODY CORPORATE, Denver, CO (US)

(72) Inventor: Jeffrey Olson, Denver, CO (US)

(73) Assignee: THE REGENTS OF THE UNIVERSITY OF COLORADO, A BODY CORPORATE, Denver, CO (US)

(21) Appl. No.: 17/601,402

(22) PCT Filed: Apr. 3, 2020
(86) PCT No.: PCT/US20/26609

§ 371 Date: Oct. 4, 2021

Related U.S. Application Data
(60) Provisional application No. 62/829,328, filed on Apr. 4, 2019.

(51) Int. Cl. A61K 9/00; A61F 9/00; A61K 31/375; A61K 31/197

(52) U.S. Cl. CPC A61K 9/0051; A61K 31/197; A61K 31/375; A61F 9/0017

(57) ABSTRACT

An intraocular antioxidant generation device is configured to be implanted within an eye. The intraocular antioxidant generation device comprises a scaffold and a generation medium coupled to the scaffold, according to various embodiments. The generation medium is configured to at least one of generate, regenerate, recycle, and produce antioxidants, according to various embodiments.

### CROSS-REFERENCE TO RELATED APPLICATIONS

This application claims priority to U.S. provisional patent application Ser. No. 62/829,328, filed on Apr. 4, 2019, the entire contents of which are incorporated herein by reference in their entirety.

### FIELD

The present disclosure relates to ocular treatment methods and implantable devices, and in particular to methods and devices for reducing intraocular oxidative damage.

### BACKGROUND

Many people suffer from various forms of retinal damage, such as glaucoma, retinal detachment, diabetic retinopathy, and macular degeneration, which can lead to diminished sight and blindness. While the severity of such ocular diseases is often age-related, oxidative damage in the eye often exacerbates disease progression. Further, oxidative damage has been demonstrated in Stargardts disease, retinitis pigmentosa, as well as other more common retinal diseases. In general, oxidative damage plays a central role in the pathogenesis of neurodegenerative diseases, including retinal dystrophies.

Additionally, oxidative damage may result in increased cell death (e.g., a chain reaction of peripheral rod photoreceptor cell death), and this increased cell death can further worsen the oxidative burden on the eye. These oxidative changes overwhelm the eye's intrinsic antioxidant mechanisms leading to production of additional reactive oxygen species and subsequent oxidative damage to the remaining cone photoreceptors. This has been further demonstrated by increased levels of oxidized glutathione in patients, signifying exhaustion of a key element of the ocular antioxidant protection system from persistent oxidative stress. Further, the crystalline lens, which is highly sensitive to oxidative damage, may undergo cataractous changes in conjunction with retinal degeneration and these changes may serve as another marker of disease severity.

Conventional treatments for oxidative damage generally include oral vitamin supplementation. The presence of antioxidants, such as ascorbic acid (e.g., vitamin C), may inhibit disease progression, and the conventional way to introduce or replenish antioxidants in the eye is via oral supplementation. However, even with a rigorous regimen of oral supplementation, often only a limited amount of antioxidant reaches the eye, and this limited amount of bioavailable antioxidant in the eye is often oxidized over time (e.g., ascorbic acid oxidizes to dehydroascorbic acid (“DHA”)).

### SUMMARY

In various embodiments, the present disclosure provides an intraocular antioxidant generation device configured to be implanted within an eye. The intraocular antioxidant generation device comprises a scaffold and a generation medium coupled to the scaffold, according to various embodiments. The generation medium is configured to at least one of generate, regenerate, recycle, and produce antioxidants, according to various embodiments.

In various embodiments, the generation medium comprises foreign cells from an animal. The foreign cells may include at least one of mitochondria, red blood cells, neutrophils, glutathione, bone cells (osteoblast, osteoclast), erythrocytes, and hepatocytes. In various embodiments, the generation medium is configured to convert dehydroascorbic acid back into ascorbic acid. In various embodiments, the generation medium is configured to convert the dehydroascorbic acid back into the ascorbic acid enzymatically. The generation medium may be configured to convert the dehydroascorbic acid back into the ascorbic acid using low molecular weight antioxidants, such as glutathione and/or cysteine.

In various embodiments, the generation medium is configured to generate ascorbic acid de novo via conversion of at least one of glucose and glycogen. In various embodiments, the scaffold comprises a crystalline structure. The scaffold may comprise an encapsulation material encapsulating the generation medium. The encapsulation material comprises a selectively permeable membrane, according to various embodiments.

Also disclosed herein, according to various embodiments, is a method of treating an ocular disorder, the method comprising implanting an intraocular antioxidant generation device into an eye. The intraocular antioxidant generation device comprises a generation medium configured to at least one of generate, regenerate, recycle, and produce antioxidants, according to various embodiments. The method may include directly delivering antioxidant intravitreally to the eye. In various embodiments, the implanting comprises positioning the intraocular antioxidant generation device such that the generation medium is disposed in fluid communication with a vitreous of the eye. Also disclosed herein, according to various embodiments, is a method of treating an ocular disorder that includes directly delivering antioxidant intravitreally to an eye.

The forgoing features and elements may be combined in various combinations without exclusivity, unless expressly indicated herein otherwise. These features and elements as well as the operation of the disclosed embodiments will become more apparent in light of the following description and accompanying drawings.

### BRIEF DESCRIPTION OF THE DRAWINGS

FIG. 1 illustrates an eye showing an implanted intraocular antioxidant generation device, in accordance with various embodiments;

FIG. 2A is schematic block diagram of an intraocular antioxidant generation device to be implanted into an eye, in accordance with various embodiments;

FIG. 2B is a depiction of an intraocular antioxidant generation device implanted, in accordance with various embodiments; and

FIGS. 3A, 3B, 3C, and 3D illustrate mass spectrometry measurements showing how an intraocular antioxidant generation device increases concentrations of antioxidant (“ascorbate”) and decreases concentrations of oxidized antioxidant (“dehydroascorbate”) over time, in accordance with various embodiments.

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements.

### DETAILED DESCRIPTION

The detailed description of exemplary embodiments herein makes reference to the accompanying drawings, which show exemplary embodiments by way of illustration. Although these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not of limitation.

The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In various embodiment, the present disclosure provides methods and devices for treatment of an ocular disorder. Generally, the disclosed method may include direct intravitreal delivery of antioxidants and/or implantation of a device that generates and/or regenerates antioxidants in situ. The term “ocular disorder” herein refers to any disease or disorder of the eye or related tissues (i.e. retina, macula, retinal blood vessels, etc.) or any symptom thereof. Non-limiting examples of ocular disorders include retinal damage, glaucoma, retinal detachment, diabetic retinopathy, retinal vascular disease, cataract, retinitis pigmentosa, and macular degeneration, among others. In addition to slowing the progression of the disease and otherwise providing the aforementioned treatment benefits, the disclosed methods and devices may specifically help patients (e.g., human or animal) have less retinal thinning, have less diminution of ERG responses, less photoreceptor atrophy, improved endogenous levels of antioxidant, and improved absorption of reactive oxygen species, according to various embodiments.

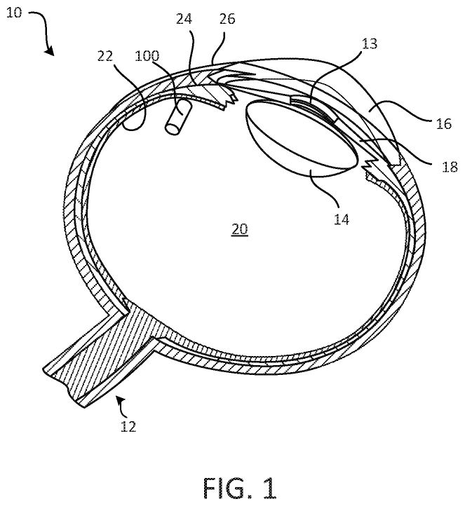

FIG. 1 illustrates an eye 10, which includes an optic nerve 12, a lens 14, a pupil 13, a cornea 16, an iris 18, a retina 22, choroid 24, sclera 26, and a vitreous 20. In various embodiments, the treatment method disclosed herein includes directly injecting antioxidants into the vitreous 20 of the eye 10, and/or implanting an intraocular antioxidant generation device 100 into the eye 10 to facilitate generation and/or regeneration of antioxidants in the vitreous 20 of the eye 10. As used herein, the term “antioxidant generation device” refers to an implantable device that is configured to generate antioxidants and/or regenerate antioxidant compounds that have been oxidized.

The intraocular antioxidant generation device 100 may be injected using a hypodermic needle, or other similar means. The intraocular antioxidant generation device 100 may be disposed within they eye 10 such that the device 100 is in fluid communication with the vitreous 20. In various embodiments, the intraocular antioxidant generation device 100 may be disposed proximate the retina 22, or may be implanted sub-retinally. In various embodiments, the intraocular antioxidant generation device 100 may be implanted through and/or anchored relative to the sclera 26, the choroid 24, or the retina 22. In various embodiments, the device 100 may be positioned in the optic nerve 12 of the eye 10. In various embodiments, the device 100 may be positioned subconjunctivally in the anterior chamber of the eye 10.

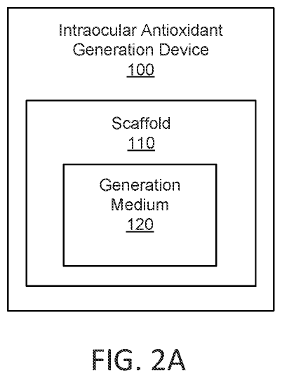

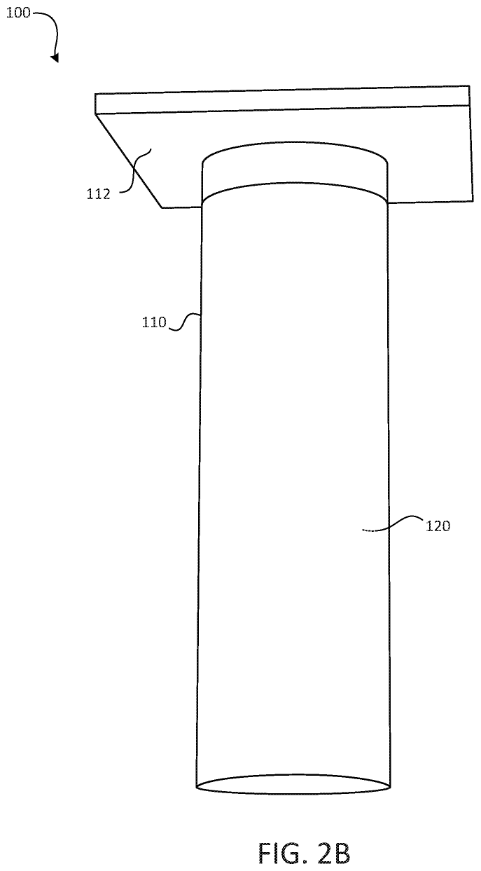

In FIG. 1, the intraocular antioxidant generation device 100 is depicted schematically, as the shape, size, and position of the intraocular antioxidant generation device 100 may vary depending on the application. That is, the depicted shape, size, and position of the device 100 does not necessarily represent an accurate or exclusive location where the implantable device 100 may be installed. Said differently, the device 100 may be implemented in various orientations/positions within the eye, and may further be implemented in other, non-ocular applications. Generally, the intraocular antioxidant generation device 100 is configured to generate or regenerate antioxidants to facilitate continued/prolonged protection of the eye 10 against oxidative damage. For example, the intraocular antioxidant generation device 100 may include a generation medium 120 (FIGS. 2A and 2B) that generates, regenerates, recycles, or otherwise outputs antioxidant compounds to the eye 10, thus vastly improving the bioavailability of such antioxidant compounds over oral supplementation, according to various embodiments.

In various embodiments, and with reference to FIGS. 2A and 2B, the intraocular antioxidant generation device 100 includes a scaffold 110 and a generation medium 120. FIG. 2A is a schematic depiction of the features/components of the antioxidant generation device 100, and FIG. 2B is an example of a physical implementation of the antioxidant generation device 100. In various embodiments, the scaffold 110 provides structural integrity to the device 100 (e.g., may have a crystalline structure) and/or the scaffold 110 may be an amorphous, membrane-type material that encapsulates and/or encases the generation medium 120. The scaffold 110 may be semi-porous, thus allowing fluid to interact with the generation medium 120. In various embodiments, the generation medium 120 may be deposited or otherwise applied onto the scaffold 110. Additional details are included below, first pertaining to the generation medium 120 and then to the scaffold 110.

In various embodiments, the generation medium 120 comprises cells or other compounds that are capable of generating, regenerating, recycling, or otherwise producing antioxidants. For example, the generation medium 120 may include foreign cells (e.g., animal cells) such as mitochondria, red blood cells, neutrophils, glutathione, bone cells (osteoblast, osteoclast), erythrocytes, and hepatocytes. The generation medium 120 may, for example, convert dehydroascorbic acid, which is the oxidized form of ascorbic acid (commonly referred to as Vitamin C), back into ascorbic acid. Thus, the generation medium 120 may be a reducing agent that facilitates reduction of oxidized antioxidant compounds back into their antioxidant form. Other types of antioxidants, such as Vitamin A, beta-carotene, Vitamin E, quercetin, etc., may be regenerated (or generated) by the medium.

In various embodiments, the generation medium 120 is specifically configured to generate or regenerate ascorbic acid. Ascorbic acid has excellent antioxidant properties, a high reactive-oxygen-species quenching ability, low toxicity, and desirable physiological effects with the vitreous. Further, ascorbic acid acts as a cofactor in the enzymatic biosynthesis of collagen, carnitine, and catecholamine and peptide neurohormones, according to various embodiments. Further, ascorbic acid generated or regenerated from the generation medium 120 also mitigates the deleterious effects of oxidants via reducing reactive oxygen and nitrogen species to stable molecules, according to various embodiments.

As mentioned above, the antioxidant effect of ascorbic acid causes the ascorbic acid to oxidize into a short-lived ascorbyl radical and then to dehydroascorbic acid (DHA). At high concentrations, DHA exerts direct cytotoxic and lethal effects, and DHA has a half-life of between about 5-7 minutes unless reduction back into ascorbate occurs. That is, if the DHA is not reduced back to ascorbic acid within a certain time frame, the DHA compound is irreversibly compromised and further degrades into diketogulonic acid which is implicated in modifying and crosslinking proteins. In various embodiments, the generation medium 120 is configured to convert DHA into ascorbic acid enzymatically via DHA reductase or nonenzymatically using low molecular weight antioxidants such as glutathione (GSH) or cysteine. In various embodiments, the generation medium 120 is made from certain human cells such as osteoblasts, erythrocytes, and hepatocytes.

In various embodiments, the generation medium 120 may produce antioxidants from other elements or compounds. The generation medium 120 may generate ascorbic acid de novo via conversion of glucose or glycogen. Ascorbic acid may be synthesized from glucose in the livers of most adult mammals, with the exception of guinea pigs, primates, humans, and others. Thus, the generation medium 120 may include cells from adult mammals that facilitate the generation of ascorbic acid. In various embodiments, the generation medium 120 may generate antioxidants from a supply of reactants that are either present in the eye 10 or that are provided with the device 100 upon being implanted within the eye 10. In various embodiments, for example, the supply of reactants may be periodically charged or otherwise re-filled in order to sustain prolonged/continued antioxidant presence in the eye 10 and thereby inhibiting oxidation damage.

In various embodiments, the scaffold 110 is the general term for the internal or external structure that provides shape and form to the antioxidant generation device 100. In various embodiments, and with specific reference to FIG. 2B, the scaffold 110 may include a flange 112 or other anchoring features that is utilized to secure the antioxidant generation device 100 in a desired position/orientation in the eye. In various embodiments, the scaffold 110 is an external encapsulation material. The encapsulation material may house, cover, enclose, or otherwise contain the generation medium 120. The encapsulation material may be a bulk housing that encases the entire device 100, or individual deposits or sections of generation medium may be individually encapsulated by the encapsulation material. In various embodiments, the encapsulation material may help prevent an adverse immune system reaction in response to implantation of the intraocular antioxidant generation device 100 within the eye 10. For example, the generation medium 120 may include foreign cells (e.g., cells not native to the human/animal whose eye within which the device is implanted), and thus the encapsulation material may prevent or at least inhibit the intraocular antioxidant generation device 100 from being rejected by the immune system of the human/animal.

In various embodiments, the encapsulation material may be a selectively permeable membrane. For example, the encapsulation material may be permeable to oxygen, carbon dioxide, glucose, antioxidants (e.g., ascorbic acid), and/or oxidized antioxidants (e.g., dehydroascorbic acid) while being impermeable to large proteins, antibodies, and other cells. In various embodiments, the encapsulation material may be made from hydroxyapatite, bone, cartilage, or other material. In various embodiments, the encapsulation material comprises a semi-porous membrane.

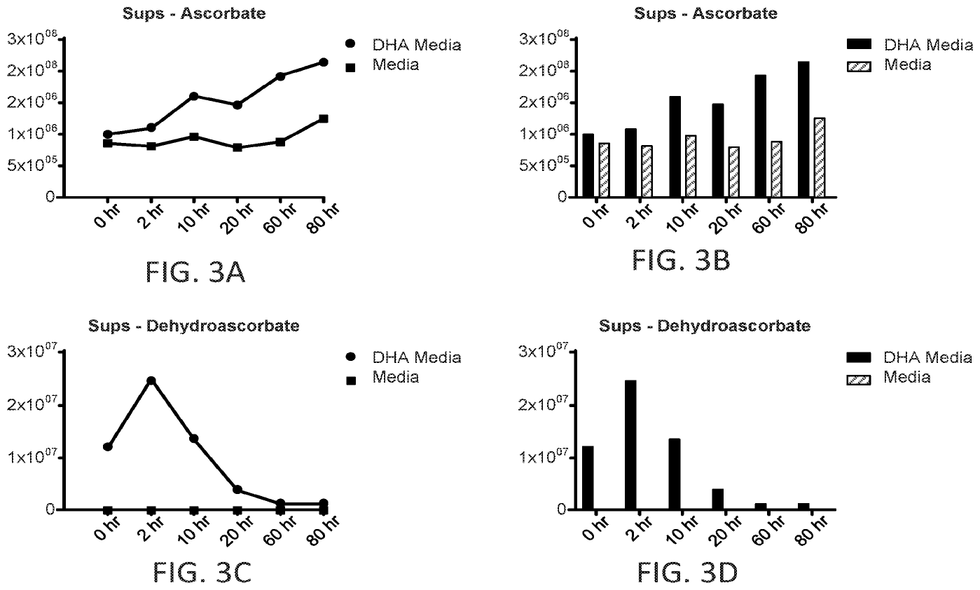

In various embodiments, and with reference to FIGS. 3A, 3B, 3C, and 3D, mass spectrometry measurements showing how the intraocular antioxidant generation device 100 increases concentrations of antioxidant (e.g., “ascorbate”) and decreases concentrations of oxidized antioxidant (e.g., “dehydroascorbate”) in the eye 10 of an animal over time. More specifically, the line graph of FIG. 3A and the bar graph of FIG. 3B both show concentration of antioxidant (e.g., “ascorbate”), as measured based off radiation absorbance levels shown along the y-axis, increasing over time along the x-axis. Similarly, the line graph of FIG. 3C and the bar graph of FIG. 3D both show concentration of oxidized antioxidant (e.g., “dehydroascorbate”), as measured based off radiation absorbance levels shown along the y-axis, decreasing over time along the x-axis.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure.

The scope of the disclosure is accordingly to be limited by nothing other than the appended claims, in which reference to an element in the singular is not intended to mean “one and only one” unless explicitly so stated, but rather “one or more.” It is to be understood that unless specifically stated otherwise, references to “a,” “an,” and/or “the” may include one or more than one and that reference to an item in the singular may also include the item in the plural. All ranges and ratio limits disclosed herein may be combined.

Moreover, where a phrase similar to “at least one of A, B, and C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

The steps recited in any of the method or process descriptions may be executed in any order and are not necessarily limited to the order presented. Furthermore, any reference to singular includes plural embodiments, and any reference to more than one component or step may include a singular embodiment or step. Elements and steps in the figures are illustrated for simplicity and clarity and have not necessarily been rendered according to any particular sequence. For example, steps that may be performed concurrently or in different order are illustrated in the figures to help to improve understanding of embodiments of the present disclosure.

Any reference to attached, fixed, connected or the like may include permanent, removable, temporary, partial, full and/or any other possible attachment option. Additionally, any reference to without contact (or similar phrases) may also include reduced contact or minimal contact. Surface shading lines may be used throughout the figures to denote different parts or areas but not necessarily to denote the same or different materials. In some cases, reference coordinates may be specific to each figure.

Systems, methods and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. An intraocular antioxidant generation device configured to be implanted within an eye, the intraocular antioxidant generation device comprising:
a scaffold; and
a generation medium coupled to the scaffold;
wherein the generation medium is configured to at least one of generate, regenerate, recycle, and produce antioxidants.

2. The intraocular antioxidant generation device of claim 1, wherein the generation medium comprises foreign cells from an animal.

3. The intraocular antioxidant generation device of claim 2, wherein the foreign cells comprise at least one of mitochondria, red blood cells, neutrophils, glutathione, bone cells (osteoblast, osteoclast), erythrocytes, and hepatocytes.

4. The intraocular antioxidant generation device of claim 1, wherein the generation medium is configured to convert dehydroascorbic acid back into ascorbic acid.

5. The intraocular antioxidant generation device of claim 4, wherein the generation medium is configured to convert the dehydroascorbic acid back into the ascorbic acid enzymatically.

6. The intraocular antioxidant generation device of claim 4, wherein the generation medium is configured to convert the dehydroascorbic acid back into the ascorbic acid using low molecular weight antioxidants.

7. The intraocular antioxidant generation device of claim 6, wherein the low molecular weight antioxidants comprise at least one of glutathione and cysteine.

8. The intraocular antioxidant generation device of claim 1, wherein the generation medium is configured to generate ascorbic acid de novo via conversion of at least one of glucose and glycogen.

9. The intraocular antioxidant generation device of claim 1, wherein the scaffold comprises a crystalline structure.

10. The intraocular antioxidant generation device of claim 1, wherein the scaffold comprises an encapsulation material encapsulating the generation medium.

11. The intraocular antioxidant generation device of claim 10, wherein the encapsulation material comprises a selectively permeable membrane.

12. A method of treating an ocular disorder, the method comprising implanting an intraocular antioxidant generation device into an eye, wherein the intraocular antioxidant generation device comprises a generation medium configured to at least one of generate, regenerate, recycle, and produce antioxidants.

13. The method of claim 12, further comprising directly delivering antioxidant intravitreally to the eye.

14. The method of claim 12, wherein the implanting comprises positioning the intraocular antioxidant generation device such that the generation medium is disposed in fluid communication with a vitreous of the eye.

15. A method of treating an ocular disorder, the method comprising directly delivering antioxidant intravitreally to an eye.
